(19) United States
(12) Patent Application Publication
(10) Pub. No.: US 2025/0325205 A1
(43) Pub. Date: Oct. 23, 2025

(54) CONTINUOUS ANALYTE METER HAVING TRANSMITTER

(71) Applicant: UXN Co., Ltd., Gyeonggi-do (KR)

(72) Inventor: Kyu Jin LEE, Seoul (KR)

(21) Appl. No.: 18/278,046

(22) PCT Filed: May 23, 2023
(86) PCT No.: PCT/KR2023/006999

§ 371 Date: Dec. 13, 2024

(30) Foreign Application Priority Data
May 23, 2022 (KR) 10-2022-0063147

(51) Int. Cl. A61B 5/1473; A61B 5/145

(52) U.S. Cl. CPC A61B 5/1473; A61B 5/14532; A61B 2560/063; A61B 2562/18

(57) ABSTRACT

Proposed is a continuous analyte meter, including an electrochemical sensor invasively inserted into the skin, a transmitter including a main substrate to which a battery is connected and a housing in which the main substrate is accommodated, the housing being attached to the skin and the main substrate controlling a signal measured by the electrochemical sensor, and a needle guiding the electrochemical sensor to be invasively inserted into the skin.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2023/006999, filed May 23, 2023, which claims priority to the benefit of Korean Patent Application No. 10-2022-0063147 filed in the Korean Intellectual Property Office on May 23, 2022, the entire contents of which are incorporated herein by reference.

### BACKGROUND

### 1. Technical Field

The present disclosure relates to a continuous analyte meter having an electrochemical sensor invasively inserted into the body to continuously measure an analyte, and a transmitter.

### 2. Background Art

When an inserter is taken as the reference position, a first end of an electrochemical sensor connected to a main substrate may be referred to as a proximal portion because it is located close to the inserter, and a second end of the electrochemical sensor inserted into the body may be referred to as a distal portion because it is located far from the inserter.

The proximal portion of the electrochemical sensor may be electrically connected to a main substrate of a transmitter, and at least a portion of the distal portion of the electrochemical sensor may be inserted into the body. The proximal portion and the distal portion may be located opposite to each other. The proximal portion of the electrochemical sensor may be electrically connected to the main substrate of the transmitter, which includes an electric circuit required to measure an analyte including glucose.

The transmitter may be placed inside the inserter along with the electrochemical sensor prior to being attached to the skin. A type in which the transmitter and the electrochemical sensor are already combined may be referred to as an all-in-one type transmitter.

The transmitter communicates a signal with the sensor. The sensor detects and measures the glucose level of a patient for a predetermined period of time, and transmits data corresponding to or related to the measured glucose level for the predetermined period of time for further analysis.

Since various electronic parts including sensors are installed in the transmitter and the transmitter is attached to the skin for a long time, an error may occur when an analyte or body fluid permeates into the transmitter.

### SUMMARY

The present disclosure is intended to propose a continuous analyte meter having a transmitter, in which a recess having an opening is provided on an outer surface of the transmitter so that a needle and a portion of an electrochemical sensor are disposed to be exposed through the recess. The transmitter according to the present disclosure does not have a through-hole through which the needle penetrates into and is removed out of the skin of the body, so the transmitter can be easily waterproofed, and the needle can be moved without frictional resistance when removed out of the skin.

A continuous analyte meter according to the present disclosure may include: an electrochemical sensor invasively inserted into the skin; a transmitter including a main substrate to which a battery is connected and a housing in which the main substrate is accommodated, the housing being attached to the skin and the main substrate controlling a signal measured by the electrochemical sensor; and a needle guiding the electrochemical sensor to be invasively inserted into the skin.

Here, the transmitter or the needle may be moved from a first position to a second position to penetrate the user's skin, a proximal portion of the electrochemical sensor may be electrically connected to the main substrate, a distal portion of the electrochemical sensor may be inserted into the body along with the needle, and a through-hole having a closed curve shape and through which the needle passes may not be formed on upper and lower surfaces of the housing.

Conventionally, through-holes for a needle are formed in upper and lower covers of a transmitter. Upon insertion, the needle sequentially passes through the through-hole formed in the upper cover and the through-hole formed in the lower cover of the transmitter, and upon removal, the needle passes through the through-holes in the reverse order and is separated from the transmitter. An electrochemical sensor is exposed through the through-holes to align with the needle.

Conventionally, an outer sealing member may be required at a junction of the upper cover and the lower cover located on the outer periphery of the transmitter. An inner sealing member may be required at a junction of the upper cover and the lower cover located between the through-holes. A sensor sealing member may be required at a portion where the electrochemical sensor is exposed through the through-holes. The present disclosure eliminates the provision of the through-holes through which the needle passes, so one outer sealing member is enough and an inner sealing member may not be required.

A conventional pillar structure may form inner and outer walls of the through-holes as walls around the through-holes. In the present disclosure, the pillar structure of the upper cover and the lower cover for forming the through-holes is also not required.

The present disclosure eliminates the provision of the pillar structure. When the provision of the conventional through-holes and the conventional pillar structure for forming the inner and outer walls of the through-holes is eliminated, an inner space of the transmitter can be greatly extended compared to a transmitter having the same diameter. The extended space enables a large-capacity installation of a battery, thereby extending the lifespan of the battery and reducing a user's cost burden. Meanwhile, it is also possible to realize an optimized arrangement of electronic parts and secure a degree of freedom in the design of a main substrate, thereby contributing to performance improvement.

For a conventional coin-sized transmitter, arranging electronic parts such as a battery and a communication unit while avoiding through-holes may cause inefficiency in circuit design, and may even cause defects in electrical connection between a proximal portion of the sensor and the main substrate.

In the present disclosure, by eliminating the provision of the through-holes and the pillar structure therearound, it is possible to significantly improve part arrangement and electrical connectivity.

Conventionally, since the sensor needs to be exposed through the narrow through-holes, it may be difficult to assemble the sensor to the transmitter. The sensor may be abruptly bent from the through-holes.

In the present disclosure, a sensor exposure hole for exposing the sensor is located in a recess of a transmitter, so the sensor can be exposed with a gentle curvature through the recess corresponding to the outer periphery of the transmitter. The sensor exposed through the recess can be advantageously bent in a gentle curve from the wide recess without needing to be abruptly bent, unlike the case of the conventional narrow through-holes. In the present disclosure, by exposing the sensor to the outside without bending the bending portion of the sensor too much, it is possible to eliminate misalignment of the sensor in a horizontal assembly position on a transmitter assembly line, misalignment of the sensor in a vertical position, and lateral and vertical bending defects of the bending portion of the sensor.

When the sensor is exposed around the conventional through-holes, there is a problem in that a waterproofing measure needs to be taken for a sensor exposure hole in a sharply curved portion. The sensor exposure hole having a sharp curve shape may cause a sealing defect. In the present disclosure, since the provision of the through-holes is eliminated and the sensor exposure hole is formed in the recess of the transmitter corresponding to the outer periphery of the transmitter, it is enough to install a sealing member in a gently curved portion. In the present disclosure, it is possible to improve close contactness of the sealing member with the sensor exposure hole. It is also possible to prevent a defect in which the sealing member protrudes from the through-holes to the outside.

In the present disclosure, eliminating the provision of the through-holes allows freedom in the design of the outer periphery of the transmitter. Since the transmitter is attached to the body, it is required to continuously check an insertion state of the sensor regardless of a user's activity. When it is possible to visually check the insertion state of the sensor even when the measurement data is out of an expected range, user satisfaction can be obtained.

It is impossible to visually observe the insertion state of the sensor into the skin through the conventional narrow through-holes. However, in the present disclosure, an insertion position of the sensor into the skin is formed on the outer periphery of the transmitter or in the recess of the transmitter, so it is easy to visually check the insertion state of the sensor. It is also possible to check whether body fluid or blood is leaking or inflammation has occurred at the site where the sensor is inserted.

The needle cannot be supported in contact with the conventional through-holes. This is because the needle needs to Alignment of the through-holes be moved in the through-holes. and the needle may be achieved by a needle handle that is supported in contact with the through-holes. The needle handle is a portion with a thick diameter at the top of the needle. However, since the diameter of the needle handle becomes smaller as the diameter of the through-holes becomes smaller, and the through-holes and the needle handle are aligned within a small diameter, a supporting force for supporting the needle in position without shaking upon insertion of the needle into the skin may be weak. This may be directly related to pain felt by a patient upon insertion of the needle. As the diameter of the through-holes becomes smaller, shaking of the needle may be increased, a contact/support force between the needle handle and the through-holes may be weakened, and shaking of the needle and patient's pain may be increased.

In the present disclosure, the wide-diameter recess and the thick-diameter needle handle can be brought into contact with each other at three or more points, so it is possible to improve the contact force/supporting force between the recess and the needle handle. It is also possible to improve accuracy of positional alignment, reduce shaking of the needle, and reduce patient's pain. In the present disclosure, it is possible to facilitate concentric alignment of the sensor already assembled in the transmitter and the needle moved vertically relative to the transmitter.

In addition, since the needle handle is supported by the recess in the transmitter, when the needle is inserted into the skin while the transmitter and the needle are lowered, a holding force of the recess holding the needle handle can be also improved. Thus, the needle with the improved holding force can puncture the skin vertically when a needle tip collides with the skin. The vertical lowering of the needle relative to the skin can greatly help relieve pain.

### BRIEF DESCRIPTION OF THE DRAWINGS

FIG. 1 is a sectional side view illustrating an inserter and a transmitter according to the present disclosure.

FIG. 2 is an assembled perspective view illustrating an electrochemical sensor and a needle according to an embodiment of the present disclosure.

FIG. 3 is a plan view of FIG. 2.

FIG. 4 is an assembled perspective view illustrating an electrochemical sensor and a needle according to another embodiment of the present disclosure.

FIG. 5 is a plan view of FIG. 4.

FIG. 6 is a plan view illustrating the arrangement of the transmitter and the needle according to the present disclosure.

FIG. 7 is a plan view illustrating a state in which another type of electrochemical sensor is disposed in the state of FIG. 6.

FIG. 8 is a plan view illustrating the needle disposed in a different direction in the transmitter according to the present disclosure.

FIG. 9 is a plan view illustrating a state in which another type of electrochemical sensor is disposed in the state of FIG. 8.

FIG. 10 is a plan view illustrating the needle disposed in an undesirable state in the transmitter according to the present disclosure.

FIG. 11 is a plan view illustrating a state in which another type of electrochemical sensor is disposed in the state of FIG. 10.

FIG. 12 is a view illustrating a relationship between an alignment position of the electrochemical sensor and an alignment position of the needle in a recess of the transmitter according to the present disclosure.

FIG. 13 is a view illustrating a dimensional relationship between an exposed portion of the electrochemical sensor provided in the transmitter and the needle according to the present disclosure.

FIG. 14 is an enlarged view illustrating a state in which a waterproof member is provided in the transmitter according to the present disclosure.

FIG. 15 is a view illustrating a case in which a plurality of recesses are formed as an embodiment of the present disclosure.

### DETAILED DESCRIPTION

Hereinafter, a case in which an electrochemical sensor 400 according to the present disclosure is used in a continuous glucose monitoring system (CGMS) for measuring the concentration of glucose in interstitial fluid or blood will be described as

However, a continuous analyte meter according to an example. the present disclosure is not limited to measuring the concentration of glucose in the body and can be extensively applied to continuous analyte meters that measure other bio-markers.

### Inserter

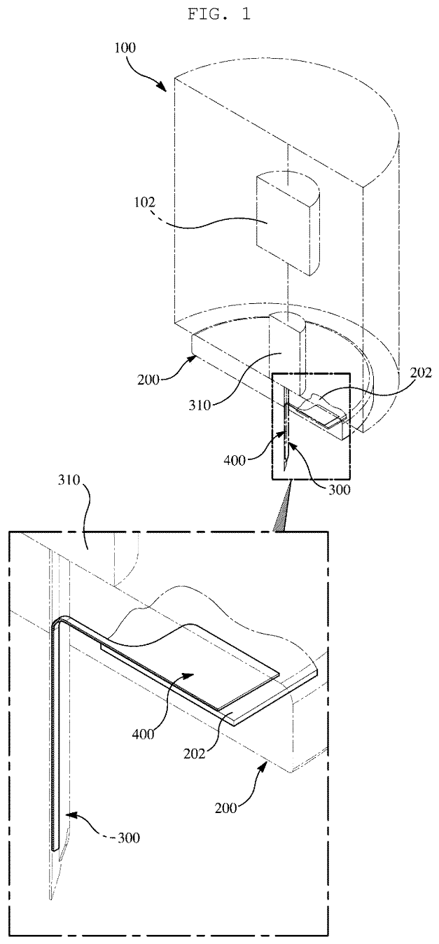

Referring to FIG. 1, the electrochemical sensor 400

according to the present disclosure may be attached to the skin along with a transmitter 200. The transmitter 200 may control a signal measured by the electrochemical Sensor 400 and continuously transmit a measured blood glucose level to an external terminal including a mobile phone.

The external terminal may be provided separately from the transmitter 200 attached to the skin, and continuously receive measurement data of the electrochemical sensor 400 wirelessly from the transmitter 200. A user may continuously monitor and diagnose measurement data of the electrochemical sensor 400 for bio-markers including glucose, lactate, and the like.

The electrochemical sensor 400 and the transmitter 200 may be provided to the user in a state of being loaded in an inserter 100 before being attaching to the skin. By a user's attachment motion, the electrochemical sensor 400 and the transmitter 200 may be detached from the inserter 100 and attached to the skin.

A first end of the electrochemical sensor 400 connected to an electrical part of the transmitter 200 including a main substrate 202 may be referred to as a proximal portion 402, a second end of the electrochemical sensor 400 at least partially invasively inserted into the body may be referred to as a distal portion 406, and a portion that connects the proximal portion 402 and the distal portion 406 to each other, is disposed between the proximal portion 402 and the distal portion 406, and is flexibly bendable may be referred to as a bending portion 405.

Invasive insertion as used herein may refer to inserting at least a portion of the distal portion 406 of the electrochemical sensor 400 into the body.

The transmitter 200 and the electrochemical sensor 400 may be provided to the user in a state in which they are already connected to each other prior to being attached to the skin.

The transmitter 200 may be located in a first position in a state of being loaded in the inserter 100. The transmitter 200 may be moved from the first position to a second position by a user's motion. The transmitter 200 may be attached to the skin in the second position. An insertion direction of the transmitter 200 and the electrochemical sensor 400 may refer to a direction from the first position to the second position.

A needle 300 may have an exposed portion exposed in the longitudinal direction thereof. A portion of the electrochemical sensor 400 may be disposed inside the needle 300. The needle 300 may serve to incise the skin and guide the electrochemical sensor 400 so that at least a portion of the distal portion 406 is invasively inserted into the body along the insertion direction.

The inserter 100 may include an actuator 102 that operates the transmitter 200 and the electrochemical sensor 400 from the first position to the second position or returns the needle 300 from the second position to a third position.

The actuator 102 may advance the needle 300 or the transmitter 200 from the first position to the second position so that the needle 300 or the distal portion 406 is inserted into the skin.

After the transmitter 200 and the electrochemical sensor 400 are attached to the skin in the second position, the actuator 102 may retract the needle 300 from the second position to the third position so that the needle 300 is separated from the transmitter 200 and the electrochemical sensor 400.

The actuator 102 may be connected to a needle handle 310 to which the needle 300 is fixed. The needle handle 310 may be detachable from the actuator 102. The actuator 102 may include a return member to which the needle handle 310 is detachably coupled.

An inner space may be provided between an upper cover 210 and a lower cover 220 of the transmitter 200. The main substrate 202 may be seated in the inner space of the transmitter 200.

The main substrate 202 may be provided with at least one of a power supply unit such as a battery required to measure the glucose concentration by the distal portion 406, a control unit including an electric circuit, a wireless communication unit for controlling data measured by the electrochemical sensor 400 and wirelessly transmitting the data to the outside, and an operational amplifier.

The power supply unit may supply a bias voltage that can generate an electrochemical reaction of a working electrode.

A signal of an analyte measured at the distal portion 406 may be amplified by the operational amplifier.

The magnitude of an output current for a given bias on the working electrode may be a measure of the concentration of the analyte, such as glucose, in the vicinity of an electrode 424.

The control unit including the electrical circuit may control the electrical potential between the working electrode and a reference electrode at one or more preset values.

A first surface of the electrochemical sensor 400 on which a sensor pad 428 is formed may face the main substrate 202, and a second surface of the electrochemical sensor 400 may be exposed to the inner space of the transmitter 200.

The sensor pad 428 may be formed at the proximal portion 402 of the electrochemical sensor 400. A contact pad electrically connected to the sensor pad 428 may be formed on the main substrate 202.

Since at least a portion of the electrochemical sensor 400 is invasively inserted into the skin, the electrochemical sensor 400 or a base layer 410 may be flexible to relieve pain during invasive insertion and reduce discomfort during wearing.

The distal portion 406 of the electrochemical sensor 400 may be disposed at the exposed portion of the needle 300 exposed along the longitudinal direction thereof. An end of the needle 300 may be in a more protruding position than an end of the distal portion 406. The distal portion 406 of the electrochemical sensor 400 may be inserted into the body after the skin is incised by the needle 300.

Pain relief and discomfort reduction are key performances of the continuous analyte meter from the user's point of view. To this end, the electrochemical sensor 400 has flexibility that it is impossible to penetrate the skin alone, and the electrochemical sensor 400 is thin and flexible enough to be inserted into the body only after the needle 300 incises the skin.

### Needle and Electrochemical Sensor

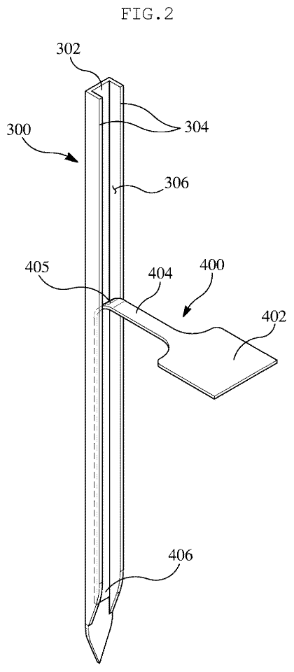

The arrangement relationship between the needle 300 and the electrochemical sensor 400 will be described with reference to FIGS. 2 to 5.

The needle 300 may have an open portion 306 exposing the inside of the needle 300 to the outside and extending along the longitudinal direction of the needle 300. A portion of the distal portion 406 or the bending portion 405 may be attached to or face the needle 300 so as to be located inside the open portion 306 upon invasive insertion into the body.

The distal portion 406 and the proximal portion 402 may lie on different planes having a predetermined angle. A bending direction of the bending portion 405 may coincide with a direction in which the inside of the needle 300 is exposed to the outside by the open portion 306.

A portion where the proximal portion 402 is electrically connected to the transmitter 200 may be located in a direction in which the inside of the needle 300 is exposed to the outside by the open portion 306.

For example, the distal portion 406 may be inserted orthogonally to the skin surface to reduce pain and discomfort. When the main substrate 202 is positioned parallel to a bottom surface of the transmitter 200, the proximal portion 402 may be positioned parallel to the main substrate 202, and the proximal portion 402 may be positioned parallel to the skin surface. In this case, the proximal portion 402 parallel to the skin and the distal portion 406 orthogonal to the skin may lie on different planes orthogonal to each other. The bending portion 405 may be bent along a direction in which the inside of the needle 300 is exposed to the outside.

The needle 300 may include a central wall portion 302 guiding invasive insertion of the electrochemical sensor 400, and opposite sidewall portions 304 preventing the electrochemical sensor 400 from being separated from the needle 300 during invasive insertion.

The central wall portion 302 may prevent the distal portion 406 or the bending portion 405 from protruding in a first axial direction. The first axial direction may refer to a direction in which the inside of the needle 300 is exposed to the outside. When the distal portion 406 or the bending portion 405 protrudes in the first axial direction, the electrochemical sensor 400 may be buckled as a protruding portion thereof is caught on the skin, and only the needle is inserted into the skin but the electrochemical sensor 400 may be come out of the skin.

The sidewall portions 304 may prevent a portion of the distal portion 406 or the bending portion 405 from being separated in a second axial direction. The second axial direction may refer to a direction orthogonal to the first axial direction. The first axial direction, the second axial direction, and the insertion direction may correspond to the axes of an orthogonal coordinate system.

The sidewall portions 304 may be disposed to have a predetermined angle with the central wall portion 302. The predetermined angle may be an angle within a range of 0 to 180 degrees with respect to surfaces of the sidewall portions 304 facing the electrochemical sensor 400.

The inner space of the needle 300 surrounded by the central wall portion 302 and the sidewall portions 304 may communicate with the outside through the open portion 306.

The electrochemical sensor 400 may have a flat plate shape. The electrode 424 of the distal portion 406 may be disposed on one or opposite surfaces of a flat plate portion.

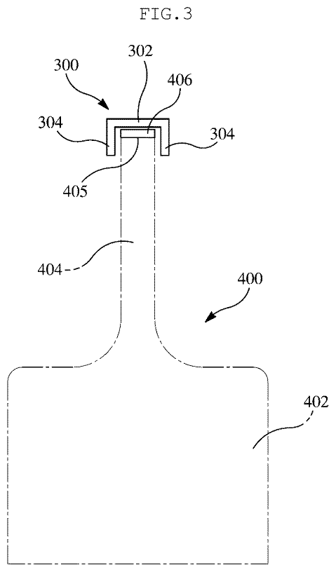

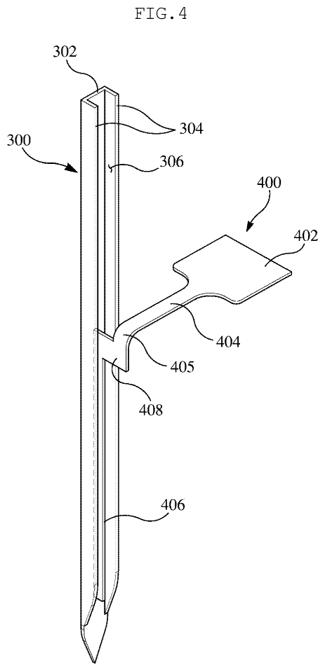

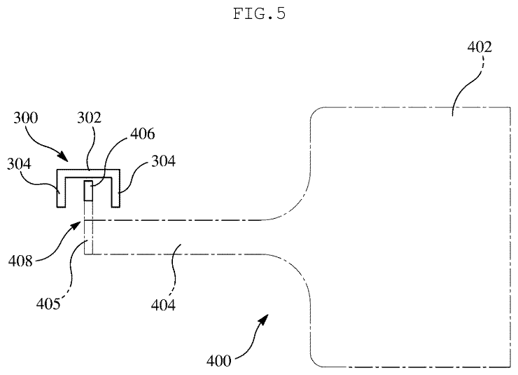

The first embodiment illustrated in FIGS. 2 and 3 may be a case in which the central wall portion 302 faces the electrochemical sensor 400 in parallel. The second embodiment illustrated in FIGS. 4 and 5 may be a case in which the central wall portion 302 faces the electrochemical sensor 400 orthogonally.

In the case of the first embodiment, the electrode 424 may make large-area contact with the outside through the open portion 306. The bending portion 405 may be bent without twisting or changing direction. In the case of the first embodiment, the bending portion 405 may be bent only one time. In this case, a torsional load applied to the bending portion 405 may be low, thereby reducing stress required to maintain a bent state.

In the case of the second embodiment, the electrochemical sensor 400 may be bent while twisting or changing in direction. The intermediate portion 404 may be bent while twisting a plurality of times or changing in direction a plurality of times so that the intermediate portion 404 extends to the proximal portion 402 while avoiding the sidewall portions 304 of the needle 300.

A side extension portion 408 may be formed so that the number of twists of the intermediate portion 404 twisting until reaching the main substrate 202 is reduced and the sidewall portions 304 of the needle 300 being raised is not caught by the intermediate portion 404 or the proximal portion 402 of the electrochemical sensor 400.

The side extension portion 408 may be a portion extending from the intermediate portion 404 in the first axial direction between the distal portion 406 and the proximal portion 402. The intermediate portion 404 adjacent to the distal portion 406 may lie on the same plane as the distal portion 406. Thus, the side extension portion 408 may be a portion extending from the intermediate portion 404 on the same plane as the distal portion 406 in the first axial direction in which the inside of the needle 300 is exposed to the outside.

In the case of the second embodiment, notches may be formed by partially cutting the intermediate portion 404 at positions adjacent to the bending portion 405. This is to minimize twisting or bending of a portion of the intermediate portion 404 with respect to the bending portion 405.

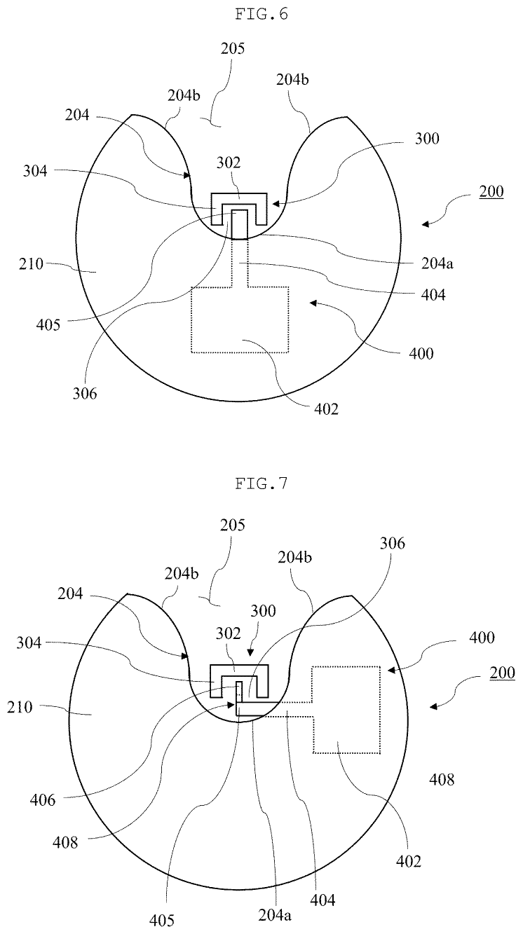

Referring to FIGS. 6 to 15, the transmitter 200 may include a housing including the upper cover 210 and the lower cover 220. The substrate 202 may be provided inside the housing. The lower cover 220 may be attached to the user's skin.

According to the greatest feature of the present disclosure, a through-hole having a closed curve shape and through which the needle 300 passes is not formed on upper and lower surfaces of the housing.

A recess 204 forming a partially open curved surface rather than a closed curved surface may be formed at one side of each of the upper cover 210 and the lower cover 220. The type forming the closed curved surface may be a through-hole (or hole), and this type of through-hole may be a structure not provided in the transmitter 200 according to the present disclosure. Thus, the transmitter 200 may have a shape in which there is no through-hole through which the needle 300 passes is formed on any surface (side surface, upper surface, and lower surface) of the housing.

An opening 205 may be formed in a portion of the recess 204.

The recess 204 may include an inner surface portion 204a corresponding to an opposite side of the opening 205 and formed at an inner position, and opposite side surface portions 204b corresponding to opposite sides of the opening 205.

The opening 205 may be where a portion of the outer periphery of the housing is cut. The opening 205 preferably has an open length small enough to prevent the needle handle 310 from passing therethrough. The needle handle 310 may have a plurality of contact points with respect to the recess. Thus, when the needle and the transmitter are lowered and the needle is inserted into the skin, the recess may suppress shaking of the needle handle so that a hole punctured in the skin matches the diameter of the needle, thereby relieving pain. The contact points of the needle handle 310 may be located around the opening 205 having a small open length.

The opening 205 may have a width narrow enough to allow the needle 300 to be moved only in the vertical direction due to contact support of the recess 204 but not to be moved away through the opening 205 in the horizontal direction. Thus, the width of the central wall portion 302 of the needle 300 may be larger than that of the opening 205.

In the case where the width of the opening 205 is smaller than that of the needle handle 310 as described above, even when the needle handle 310 provided on top of the needle 300 is moved as the needle 300 is moved, the side surface portions 204b of the recess 204 and the opening 205 may serve to guide the needle to be smoothly moved without shaking.

On the other hand, as another embodiment, in the case where the open length (length in the circumferential direction) of the opening 205 is larger than that of the needle handle 310, a space in which the needle 300 can be moved more smoothly without any interference may be secured. Thus, the space may accommodate an error that may occur between a movement position of the needle 300 and a set position so that the needle 300 is moved without any frictional resistance.

The inner surface portion 204a and the side surface portions 204b constituting the recess 204 may all form one surface of the housing constituting the transmitter 200, and may correspond to the outer periphery of each of the upper cover 210 and the lower cover 220.

In addition, each of the inner surface portion 204a and the side surface portions 204b of the recess 204 may be in the form of a curved surface or a flat surface, and may be continuously formed.

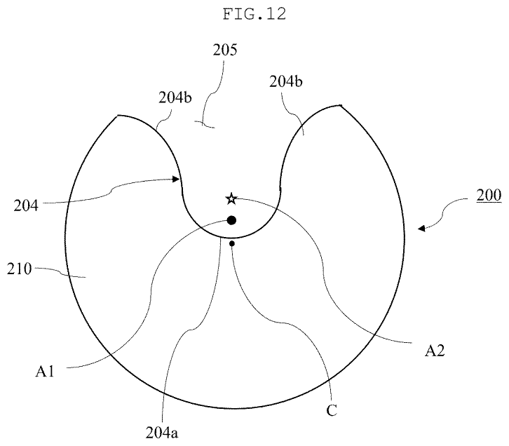

Referring to FIG. 12, in the case where the housing of the transmitter 200 has a circular shape when viewed from top, the position of the inner surface portion 204a of the recess 204 may be eccentric from a center C.

When the recess 204 is eccentric from the center of the transmitter 200, it may be easy to secure an installation space for the substrate 202 or electronic parts provided inside the transmitter 200, and it may be advantageous to miniaturize the transmitter 200.

Since the electrochemical sensor 400 according to the present disclosure has a thin film thickness of equal to or less than 100 micrometers, when the sensor is provided inside the transmitter 200, it may not occupy a large volume of space and thus contribute to slimming of the transmitter 200.

A portion of the electrochemical sensor 400 provided inside the transmitter 200 may be disposed to be exposed to the outside of the transmitter 200. The proximal portion 402 and a portion of the intermediate portion 404 of the electrochemical sensor 400 (hereinafter referred to as ‘embedded portion’) may be disposed inside the transmitter 200, and a portion of the intermediate portion 404, the bending portion 405, and the distal portion 406 (hereinafter referred to as ‘exposed portion’) may be disposed outside the transmitter 200.

The exposed portion disposed outside the transmitter 200 may be positioned in the recess 204. Here, the exposed portion of the electrochemical sensor 400 and the needle 300 may face each other in order to insert the electrochemical sensor 400 into the user's skin.

In more detail, the distal portion 406 of the electrochemical sensor 400 may be disposed to face the inside of the central wall portion 302 of the needle 300 through the open portion 306 of the needle 300.

In the present disclosure, it may be preferable that the needle 300 is not freely disposed when moved along the recess 204 of the transmitter 200 but is disposed at a specific position.

That is, the needle 300 may include the central wall portion 302 having a predetermined length, the opposite sidewall portions 304 bent and protruding from opposite sides of the central wall portion 302, and the open portion 306 formed at a position facing the central wall portion 302 and between the sidewall portions 304. The open portion 306 may face a direction opposite to the opening 205 of the recess 204 formed in the transmitter 200. When the open portion faces the sensor, the sensor may be prevented from being caught on the sidewall portions of the needle being moved.

Here, the bending portion 405 and the distal portion 406 of the electrochemical sensor 400 may be disposed in a state of being inserted in the inner space between the sidewall portions 304 of the needle 300.

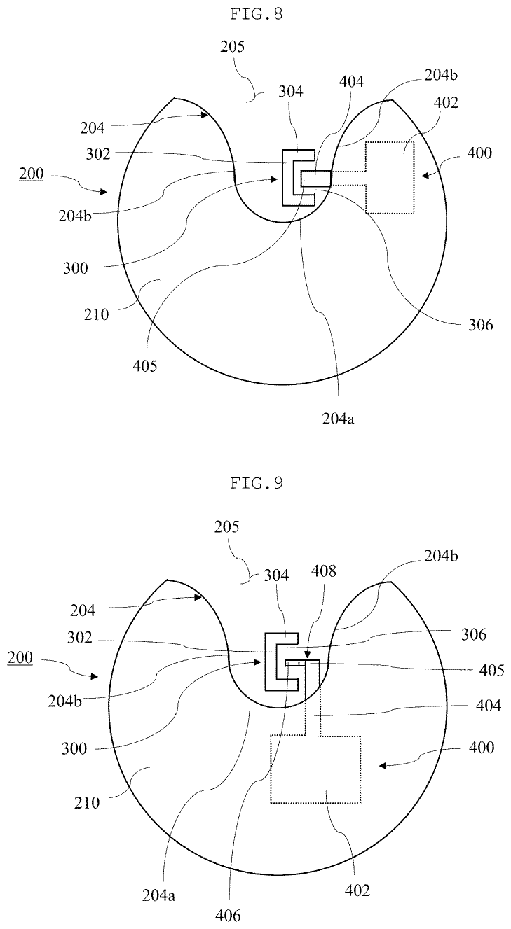

As illustrated in FIG. 8, another arrangement of the needle 300 may be such that the open portion 306 of the needle 300 faces one side surface portion 204b of the side surface portions 204b formed at opposite sides of the housing. In this case, the arrangement position of the electrochemical sensor 400 may also vary. The electrochemical sensor 400 guided by the needle 300 may be applied to both the type illustrated in FIG. 2 and the type illustrated in FIG. 4.

Thus, the open portion 306 of the needle 300 may be disposed to face either the inner surface portion 204a or any one of the side surface portions 204b of the recess 204, and accordingly, two types of electrochemical sensors 400 may be disposed correspondingly.

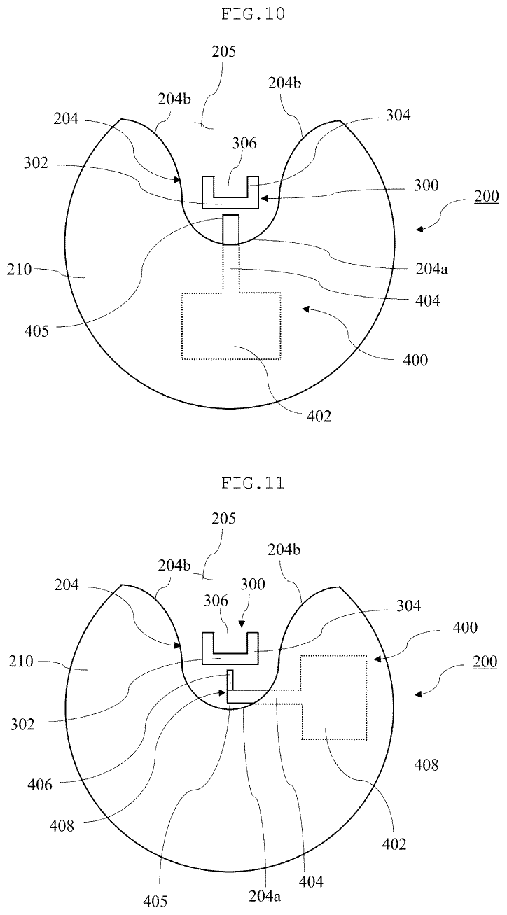

On the other hand, as illustrated in a comparative embodiment of FIG. 10, in the case where the open portion 306 of the needle 300 is not disposed to face either the inner surface portion 204a or any one of the side surface portions 204b of the recess 204 but is disposed to face the opening 205, the exposed portion of the electrochemical sensor 400 cannot be guided and inserted into the user's skin. This type of comparative embodiment is not desirable.

As in the comparative embodiment of FIG. 10, when the open portion 306 of the needle 300 is disposed to face the opening 205 of the recess 204, the central wall portion 302 of the needle 300 cannot face the exposed portion of the electrochemical sensor 400. The exposed portion of the electrochemical sensor 400 cannot be inserted into the space between the sidewall portions 304 formed at opposite sides of the central wall portion 304. The needle 300 cannot guide and insert the exposed portion of the electrochemical sensor 400 into the skin.

Thus, the arrangement state of the needle 300 illustrated in FIGS. 10 and 11 may not be desirable.

In addition, the present disclosure may have a structure that does not require a separate guide member inside the transmitter 200 to facilitate stable movement of the needle 300. The guide member inside the transmitter 200 may include, for example, a cylindrical structure having a through-hole. By eliminating the provision of this unnecessary guide member, the inner space of the transmitter 200 may be used more extensively.

The needle 300 is designed to essentially pass through the recess 204 of the transmitter 200 during movement, and the recess 204 is a structure formed outside the transmitter 200.

In addition, according to the present disclosure, when an alignment position of the bending portion 405 of the electrochemical sensor 400 disposed in the recess 204 is referred to as a first alignment position A1 and an alignment position of the needle 300 is referred to as a second alignment position A2, the first alignment position A1 and the second alignment position A2 may be the same positions.

In the first alignment position A1, the electrochemical sensor 400 may be disposed in the inner space of the needle 300 composed of the central wall portion 302 and the sidewall portions 304 formed at opposite sides of the central wall portion 302. That is, in the first alignment position A1, the sensor may be disposed in a state of being surrounded by the central wall portion 302 and the sidewall portions 304 of the needle 300.

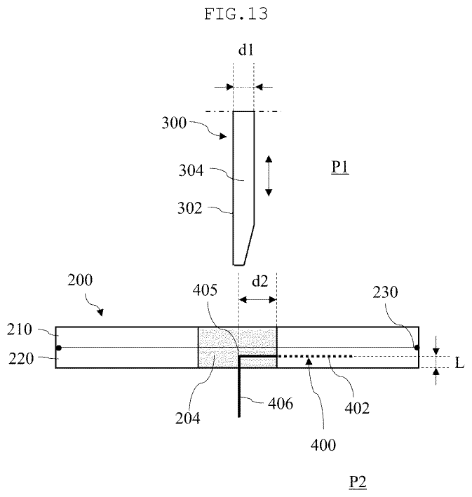

In addition, referring to FIG. 13, when a protruding length of the sidewall portions 304 of the needle 300 is referred to as a first length d1 and a length from a position where the electrochemical sensor 400 starts to be exposed from the inside of the transmitter 200 to the outside to the bending portion 405 is referred to as a second length d2, the second length d2 may be longer than the first length d1. Thus, a portion of the electrochemical sensor 400 corresponding to the second length d2 may be disposed to protrude from the needle 300. Here, when the bonding portion 405 is disposed in the inner space of the needle 300, an insertion depth of the electrochemical sensor 400 inserted into the skin may be advantageously increased.

In addition, the electrochemical sensor 400 may be provided in a state of being spaced apart from an inner bottom portion of the transmitter 200 by a separation distance L.

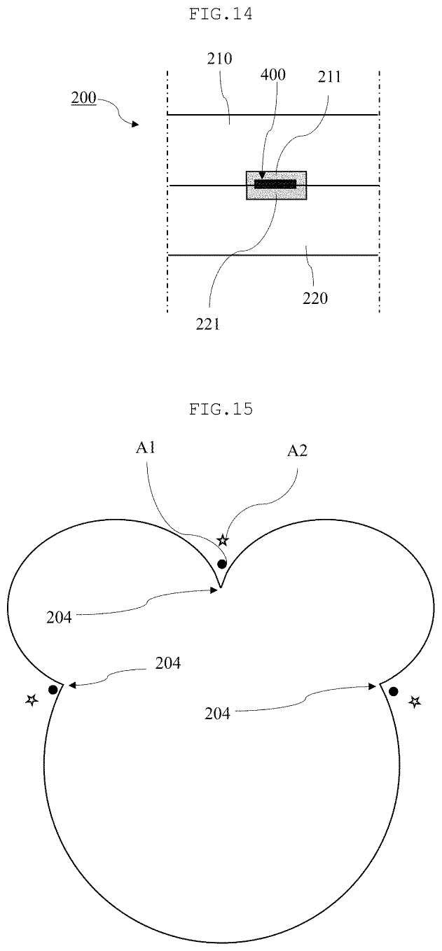

Referring to FIG. 14, the transmitter 200 needs to be kept in a waterproof state. In particular, a portion of the housing constituting the transmitter 200 where the electrochemical sensor 400 is exposed to the outside of the housing is required to be waterproofed.

When the housing is composed of the upper cover 210 and the lower cover 220, a waterproof member may be provided at each of the upper cover 210 and the lower cover 220. When the upper cover 210 and the lower cover 220 are coupled to each other in a state in which a portion of the electrochemical sensor 400 is exposed, the respective waterproof members and the exposed portion of the electrochemical sensor 400 may be brought into close contact with each other to be waterproofed. Since the position where the electrochemical sensor 400 is exposed corresponds to the outer peripheries of the upper cover 210 and the lower cover 220, the waterproof members may be installed on gently curved portions, thereby improving sealing performance.

The waterproof members may include a tape. In an embodiment of the present disclosure, tapes 211 and 221 for waterproofing may be attached to the upper cover 210 and the lower cover 220, respectively, and may be brought into close contact with the exposed portion of the electrochemical sensor 400. The tapes 211 and 221 may be attached in multiple layers to increase close contactness with the exposed portion of the electrochemical sensor 400.

A packing member 230 such as an O-ring or a rubber ring may be provided to seal the remaining portion of the outer periphery of the housing except for the portion where the portion of the electrochemical sensor 400 is exposed from the housing. Since there is no through-hole in the transmitter and there is only one junction on the outer periphery thereof, all junctions of the upper cover 210 and the lower cover 220 may be waterproofed with only one O-ring.

When a position spaced apart from the upper cover 210 and the lower cover 220 constituting the transmitter 200 is referred to as a first position P1 and a position after movement along the recess 204 of each of the upper cover 210 and the lower cover 220 is defined as a second position P2, the needle 300 may be separated from the transmitter 200 while being moved from the second position P2 to the first position P1 through the recess 204 of the transmitter 200.

An upper surface of the housing may face the inserter, a lower surface of the housing may face the skin, and a side surface of the housing may be open. The recess opening the side surface of the housing may be formed in the transmitter. The needle or sensor may be visually observed through the recess in the side surface of the transmitter.

What is claimed is:

1. A continuous analyte meter comprising:
an electrochemical sensor invasively inserted into the skin;
a transmitter including a main substrate to which a battery is connected and a housing in which the main substrate is accommodated, the housing being attached to the skin and the main substrate controlling a signal measured by the electrochemical sensor; and
a needle guiding the electrochemical sensor to be invasively inserted into the skin,
wherein the transmitter or the needle is moved from a first position to a second position to penetrate the skin,
a proximal portion of the electrochemical sensor is electrically connected to the main substrate,
a distal portion of the electrochemical sensor is inserted into the body along with the needle, and
a through-hole having a closed curve shape and through which the needle passes is not formed on upper and lower surfaces of the housing.

2. The continuous analyte meter of claim 1, wherein the electrochemical sensor extends from an inside to an outside of the housing through an outer periphery of a side surface of the housing.

3. The continuous analyte meter of claim 1, wherein the needle is moved along a vertical direction of the housing at a position spaced a predetermined distance from an outer periphery of the housing.

4. The continuous analyte meter of claim 1, wherein a recess is formed by recessing a side surface of the housing, and
the distal portion and the needle are disposed in the recess.

5. The continuous analyte meter of claim 1, wherein a recess is formed by recessing a side surface of the housing, and
the needle handle of the needle has a plurality of contact points with the recess.

6. The continuous analyte meter of claim 1, wherein an opening is formed on a side surface of the housing,
the needle comprises: a central wall portion; opposite sidewall portions protruding from opposite sides of the central wall portion; and an open portion formed between the sidewall portions, and
the open portion of the needle faces a direction not facing the opening.

7. The continuous analyte meter of claim 1, wherein a recess is formed in the housing,
the recess comprises: an inner surface portion; opposite side surface portions formed at opposite sides of the inner surface; and an opening formed at an opposite side of the inner surface portion,
the needle comprises: a central wall portion; opposite sidewall portions protruding from opposite sides of the central wall portion; and an open portion formed between the sidewall portions, and
the open portion of the needle faces either the inner surface portion or any one of the side surface portions of the recess.

8. The continuous analyte meter of claim 1, wherein the electrochemical sensor comprises an intermediate portion,
the intermediate portion is formed between the proximal portion and the distal portion and protrudes from an inside to an outside of the housing,
the intermediate portion is provided with a bending portion formed by bending the intermediate portion so as to form different planes, and
the bending portion is disposed in an inner space of the needle.

9. The continuous analyte meter of claim 1, wherein a recess is formed on an outer surface of the housing, and
when an alignment position of the bending portion of the electrochemical sensor disposed in the recess is referred to as a first alignment position and an alignment position of the needle is referred to as a second alignment position, the first alignment position and the second alignment position are the same positions.

10. The continuous analyte meter of claim 1, wherein the needle comprises opposite sidewall portions protruding from opposite sides thereof,
the electrochemical sensor comprises an intermediate portion formed between the proximal portion and the distal portion and protruding from an inside to an outside of the housing,
the intermediate portion is provided with a bending portion formed by bending the intermediate portion so as to form different planes, and
when a protruding length of the sidewall portion or a thickness of the needle is referred to as a first length and a length from a position where the electrochemical sensor starts to be exposed from the inside to the outside of the housing to the bending portion is referred to as a second length, the second length is longer than the first length.

11. The continuous analyte meter of claim 1, wherein the housing comprises an upper cover and a lower cover,
a waterproof member is provided at a junction on an outer periphery of the upper cover and the lower cover,
the electrochemical sensor comprises an intermediate portion protruding from an inside to an outside of the housing, and
the intermediate portion is in close contact with the waterproof member.

12. The continuous analyte meter of claim 1, wherein a recess is formed on an outer surface of the housing, and the recess is formed at a position eccentric from a center of the housing.

13. The continuous analyte meter of claim 1, wherein a plurality of recesses are formed on an outer surface of the housing, and
a plurality of protrusions of the same number as the number of the recesses are formed on the outer surface of the housing.

14. The continuous analyte meter of claim 1, wherein the upper surface of the housing faces the inserter, the lower surface of the housing faces the skin, and a side surface of the housing is open.

15. The continuous analyte meter of claim 1, wherein the upper surface of the housing faces the inserter, the lower surface of the housing faces the skin, and a recess opening a side surface of the housing is formed in the transmitter, and
the needle or the sensor is visually observed through the recess on the side surface of the transmitter.
